(19) United States
(12) Patent Application Publication
(10) Pub. No.: US 2013/0236504 A1
(43) Pub. Date: Sep. 12, 2013

(54) Delivery System for Enhancing Drug Efficacy

(71) Applicants: CLEMSON UNIVERSITY, Clemson, SC (US); MEDICAL UNIVERSITY OF SOUTH CAROLINA, Charleston, SC (US)

(72) Inventors: Frank Alexis, Greenville, SC (US); Bruce Frankel, Mt. Pleasant, SC (US)

(73) Assignees: Medical University of South Carolina, Charleston, SC (US); Clemson University, Clemson, SC (US)

(21) Appl. No.: 13/784,930

(22) Filed: Mar. 5, 2013

Related U.S. Application Data
(60) Provisional application No. 61/607,036, filed on Mar. 6, 2012.

(51) Int. Cl. A61K 31/52; A61K 31/4188

(52) U.S. Cl. CPC A61K 31/52; A61K 31/4188
USPC 424/400; 514/263.37; 544/276; 514/263.32; 424/43

(57) ABSTRACT

Disclosed are delivery systems that can be used for treating cancer. The delivery systems include a delivery vehicle in conjunction with a chemo-adjuvant. The chemo-adjuvant can enhance the efficacy of a therapeutic agent that can be delivered in conjunction with the delivery vehicle or can be delivered independently of the delivery vehicle.

### CROSS REFERENCE TO RELATED APPLICATION

The present application claims filing benefit of U.S. Provisional Patent Application No. 61/607,036 having a filing date of Mar. 6, 2012, which is incorporated herein for all purposes.

### BACKGROUND

The breakthrough potential of nanotechnology is being increasingly recognized with several first-generation non-targeted nano-carriers being FDA approved (Alexis, et al., 2008a; Davis, et al, 2008; Zhang, et al., 2008). Nanoparticles may also be surface functionalized with biomolecules for tumor targeting in order to improve the specific internalization of drugs into cancer cells. For example, glioblastoma cancer stem cell markers have been identified (Duntsch Journal of Neuro-Oncology (2005) 71: 245-255), e.g., CD133+ cancer stem cells have been implicated in gliomagenesis (Beir Cancer Res 2007; 67: (9). May 1, 2007), and can be targeted via functionalized nanoparticles. Nanoparticles can improve the therapeutic index of currently available drugs by increasing their efficacy, lowering their toxicity, and creating steady-state therapeutic levels of drugs for extended time periods (Alexis et al., 2008b; Brannon-Peppas and Blanchette, 2004; Gelperina et al., 2005; Peer at al., 2007; Pridgen at al., 2007; Wang et al., 2007).

The use of combination chemotherapeutic regimens has also been developed in an attempt to reduce drug resistance to individual agents, such as temozolomide. Unfortunately, however, these regimens often expose patients to unacceptable side effects necessitating dose reduction. For instance, glioblastoma resistance to temozolomide and other alkylating agents is due to the presence of the cellular DNA repair protein methylguanine methyltransferase (MGMT). Depletion of this DNA repair enzyme by O6-BG, a low molecular weight substrate of MGMT, increases temolozolomde induced-cytotoxicity. Systemic intravenous chemotherapy regimens of O6-BG given in combination with chemotherapeutic agents including bis-chloroethylnitrosourea, temozolomide, or Gliadel wafers have been examined in clinical trials (Rabik et al., 2006), but require cytotoxic drug dose reduction in order to prevent added toxicity.

What are needed in the art are compositions and methods that allow for the efficient delivery of a chemo-adjuvant, such as O6-benzylguanine, to increase the effect of chemotherapeutic agents. Compositions and methods that can specifically target useful agents to cancer cells while enhancing the effect of the chemotherapeutic agent would be of great benefit.

### SUMMARY

According to one embodiment, disclosed is a delivery system. The delivery system can include a chemo-adjuvant and a delivery vehicle. The chemo-adjuvant can enhance the efficacy of a therapeutic agent, for instance in treatment of a cancer, and can be provided in conjunction with the delivery vehicle.

Also disclosed are methods for utilizing the delivery system. For instance, a delivery system can be administered to an area that includes cancer cells. The method can be utilized, for instance, in treatment of a subject suffering from cancer.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the biodistribution and cytotoxicity of drug combinations: FIG. 1A illustrates the biodistribution of a combination of two toxic chemotherapeutic agents when the two agents are administered in a free distribution protocol (i.e., neither drug is conjugated with a delivery vehicle); FIG. 1B illustrates the biodistribution of the two chemotherapeutic agents when the first drug is delivered in a nanoparticle formulation and the second agent is delivered in a free distribution profile; FIG. 1C illustrates the biodistribution of delivery of a non-toxic chemo-adjuvant in conjunction with a delivery vehicle and separate free distribution of a therapeutic agent.

FIG. 2 illustrates the immunoreactivity of CD 133+ and glial fibrillary acidic protein (GFAP) in and around a glioblastoma recurrence.

FIG. 3 illustrates U138 cell viability upon incubation with various concentrations of a non-toxic drug.

FIG. 4 compares the cell viability of U-138 cells upon incubation with toxic and non-toxic drugs (1000 fold concentration compared to toxic drugs).

FIG. 5 compares the cell viability of U-138 cells upon incubation with different combinations and concentrations of toxic and non-toxic drugs.

FIG. 6 presents cell viability of U-138 cells upon incubation with toxic and non-toxic drugs including non-toxic drugs provided in a delivery system as presented herein.

### DETAILED DESCRIPTION

Reference will now be made in detail to various embodiments of the presently disclosed subject matter, one or more examples of which are set forth below. Each embodiment is provided by way of explanation, not limitation, of the subject matter. In fact, it will be apparent to those skilled in the art that various modifications and variations may be made to the present disclosure without departing from the scope or spirit of the disclosure. For instance, features illustrated or described as part of one embodiment, may be used in another embodiment to yield a still further embodiment. Thus, it is intended that the present disclosure cover such modifications and variations as come within the scope of the appended claims and their equivalents. In addition, before the present compounds, compositions, articles, devices, and/or methods are disclosed and described, it is to be understood that they are not limited to specific synthesis methods or to particular reagents, and such may, of course, vary. It is also to be understood that the terminology used herein is for the purpose of describing particular embodiments only and is not intended to be limiting.

In general, disclosed herein are delivery systems for treatment of cancers. More specifically, disclosed systems include a delivery vehicle in conjunction with a chemo-adjuvant agent, and in one embodiment, the delivery system can be specifically targeted to the cancer cells. The delivery system can be delivered as a component of a chemotherapeutic regimen and can, among other benefits, provide a solution to two specific challenges that have been present in chemotherapeutics. First, the system can be utilized to deliver a chemo-adjuvant agent with optimum release kinetics inside targeted cancer cells, which can improve efficacy of a chemotherapeutic agent utilized in conjunction with the delivery system. Secondly, the delivery of the chemo-adjuvant to the cancer cells in conjunction with the delivery vehicle can reduce the dosage and side effects of chemotherapeutic agents common in the past with chemotherapeutic drug combinations.

The use of the disclosed chemotherapeutic regimens may reduce drug resistance to individual chemotherapeutic agents, such as temozolomide, while preventing unacceptable side effects necessitating dose reduction. For example, glioblastoma resistance to temozolomide and other alkylating agents can be reduced or prevented using a chemo-adjuvant such as a non-toxic chemo-sensitizer delivered by use of the disclosed delivery systems. Moreover, a non-toxic chemo-adjuvant can be utilized instead of additional chemotherapeutic drugs as has been attempted in the past, further improving the treatment regimen over previously known systems.

In order to be effective, it is desired that a chemo-adjuvant enter cancer cells and increase the efficacy of the chemotherapeutic (e.g., an alkylating agent). To address this desire, the disclosed delivery systems include delivery of a chemo-adjuvant, such as the non-toxic chemo-sensitizer O6-Benzylguanine (O6-BG) by use of a delivery vehicle, along with co-independent delivery of a chemotherapeutic reagent.

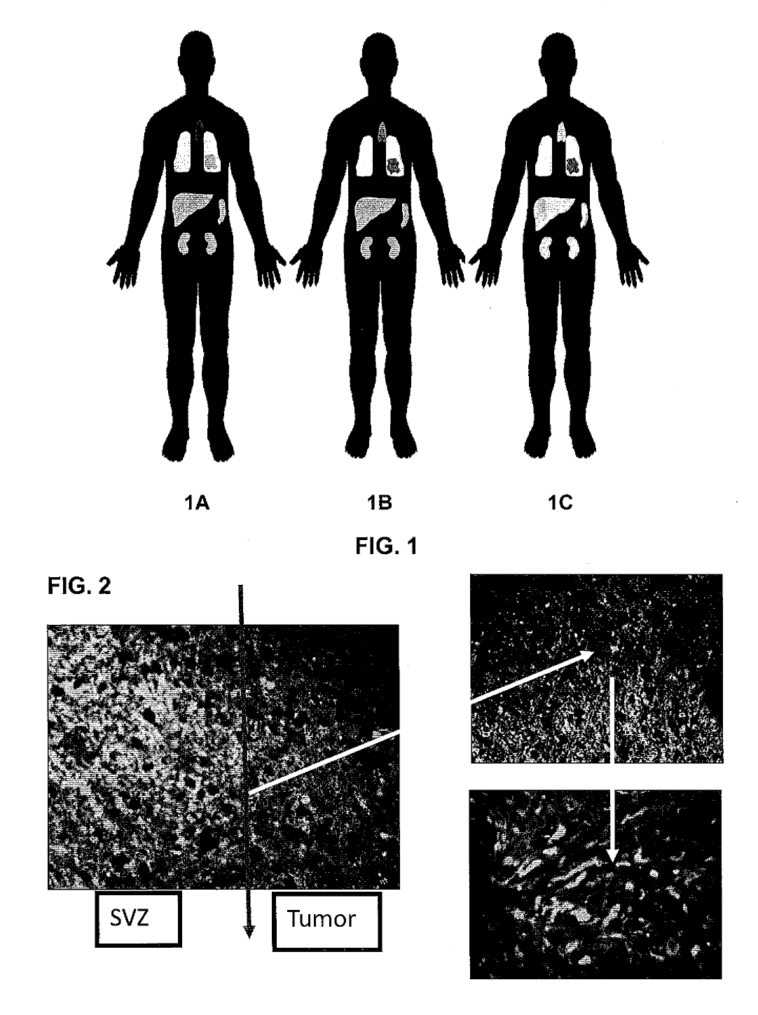

For example, and with reference to FIG. 1, in a traditional treatment protocol as illustrated in FIG. 1A two different toxic therapeutic agents will be delivered freely to a subject. The two agents can be distributed and cause cytotoxic effects in a variety of locations throughout a subject's system including, for example, the thymus, liver, lungs, kidneys, etc. In more recent methods, as illustrated in FIG. 1B, one of the therapeutic agents can be delivered in a more targeted approach, for instance by use of a delivery vehicle loaded with the therapeutic agent, and the cytotoxic effects can be less, though the therapeutic agents may still cause systemic toxic effect. In this case, both the vehicle loaded with one therapeutic agent and the second therapeutic agent are toxic. Through utilization of the disclosed delivery system, and as illustrated in FIG. 1C, a non-toxic chemo-adjuvant can be delivered by use of the delivery vehicle, and the therapeutic can be delivered either freely or in conjunction with the same or a different delivery vehicle. In this case, the vehicle loaded with one therapeutic agent is non-toxic and the second therapeutic agent is toxic. This approach can decrease cytotoxic effects of the treatment as the chemo-adjuvant can boost the effectiveness of the therapeutic at the targeted site, thereby decreasing the need of secondary therapeutics as well as the dosage of the primary therapeutic.

The delivery system can be used for treatment of cancers, such as glioblastoma cancers. The delivery systems are not limited to in vivo treatment protocols, however. For example, the delivery systems can be beneficially utilized in determination of the kinetics of the delivery, release, and/or activity of chemotherapeutic reagents, alone or in combination. In addition, the cytotoxicity of any combination of chemotherapeutic agents and chemo-adjuvants can be identified and determined. By way of example, U87 cells can be used for such determinations. Loading and release kinetics of compounds can also be determined as well as the activity of chemo-adjuvants such as non-toxic chemo-sensitizers. Moreover, a delivery vehicle can be labeled, e.g., fluorescently labeled, and the system can be used for in vivo analysis of both systemic biodistribution and tumor distribution. This type of analysis can be performed using, for example, blab/c nude mice with intracranial human brain tumor xenografts (U87 glioblastoma cells), for brain tumor analysis. This type of analysis can be used to quantify the amount of reagent accumulated in the target tissues, such as glioblastoma cells.

As mentioned, the delivery systems include a chemo-adjuvant in conjunction with a delivery vehicle. As utilized herein, the term “chemo-adjuvant” generally refers to any agent that can be added to an existing therapeutic regimen, e.g., a chemotherapeutic regimen, such that the efficacy of a therapeutic agent is enhanced and as such a cancer therapeutic regimen is enhanced. Enhancement can encompass, for example, the ability to give a therapeutic at a lower efficacious dose; an increase in efficacy of treatment; or generally increasing, enhancing or stabilizing the therapeutic effect of the cancer therapeutic treatment. While the chemo-adjuvant can provide a direct therapeutic effect in the cancer treatment, this is not a requirement, and the chemo-adjuvant is not necessarily a cancer therapeutic agent on its own.

The chemo-adjuvant can encompass, without limitation, a chemo-sensitizer, an MGMT inhibitor, a chemo-enhancing agent, a radiation sensitizer, a photosensitizer, a detoxification agent, a sensitizer agent, a chemo-protective agent, a radio-protective agent, leucovorin, derivatives of these, or any combinations thereof. For instance, the delivery vehicle can carry more than one chemo-adjuvant agent, such as 2, 3, 4, or 5 chemo-adjuvant agents. The chemo-adjuvant can generally, though not necessarily, be a non-toxic agent. As utilized herein, the term “non-toxic” generally refers to an agent that is not capable of producing detectable harm to cells or tissues in an MTT assay. More specifically, a non-toxic agent is not capable of blocking the growth of cells below 80% at 1 μM in the MTT.

As utilized herein, the term “chemo-sensitizer” generally refers to any agent that makes a cell more susceptible to a chemotherapeutic agent, such as a DNA repair inhibitor. In certain embodiments a DNA repair inhibitor can be a PARP inhibitor, ATM kinase inhibitor, base excison repair inhibitor, DNA PKCs inhibitor, and multiple target inhibitors. It can include, without limitation, a natural or synthetic compound.

An MGMT inhibitor chemo-adjuvant includes any molecule that can inhibit or reduce the activity of MGMT. MGMT is encoded by the MGMT gene, and is a protein that removes the alkylation at the O6 of guanine (O6-alkyl-guanine) in nucleic acids. MGMT removes the nucleic acid lesion by acting as an acceptor for the alkylating functional group, through stoichiometric transfer to a methyl residue of a cysteine in the protein. In glioblastoma multiforme, the methylation state of the promoter of the MGMT gene is important. In cells with high methylation, the drug temozolomide is active, and in cells with low methylation, the drug is inactive, which makes the cells less susceptible to alkylating chemotherapies, with for example, temozolomide.

An MGMT inhibitor can include, without limitation, Patrin™ (PaTrin-2, Lomeguatrib), O6-substituted compounds, O6-heteroalkylmethyl analogs O6-BG, O6-2-fluoropyridinylmethylguanine (O6FPG), O6-3-iodobenzylguanine, O6-4-bromothenylguanine, O6-5-iodothenylguanine, quinolinone derivatives, or alkylphenyl-triazolo-pyrimidine derivatives. O⁶-heteroalkylmethyl analogues of O6-methylguanine, such as the furfuryl and thenyl compounds, such as 4-bromothenyl derivatives can also be utilized. Other MGMT inhibitors include, for example, O6-BG, O6-2-fluoropyridinylmethylguanine (O6FPG), O6-3-iodobenzylguanine, O6-4-bromothenylguanine, and O6-5-iodothenylguanine with the corresponding C8-linker β-D-glucose derivatives.

A chemo-enhancing agent is any agent that enhances the activity of a therapeutic. One example of a chemo-enhancing agent would be an MGMT inhibitor, such as O6-BG. In use, delivery of a chemo-enhancing agent such as O6-BG can result in MGMT depletion, thereby increasing tumor sensitivity to alkylating agents such as temozolomide without added new side effects as have been noted with previously known drug combinations.

The chemo-adjuvant can be a chemo-protective agent, which can be any agent that protects a cell from chemotherapy. Examples of a chemo-protective agent are folate agonists, such as leucovorin, which protects cells from methotrexate (MTX). Other exemplary chemo-protective agents include dimesna (Tavocept™) (prevents neuro- and nephrotoxicity), glutathione (prevents neurotoxicity from chemotherapeutic agents working against the mitotic fuse such as vinca alkaloids (vincristine, and vinblastine) and cyclotaxans (Taxol®), thiol compounds (protect against nephrotoxicity caused by cis-platin), free radical scavengers (prevents anthracycline induced cardiotoxicity), adenosine (reduces chemotherapeutic induced bone marrow toxicity), NI-Acetylcysteine (reduces cis-platin induced toxicity), amifostine (acts as both a chemoprotectant and radiation protectant agent).

A chemo-adjuvant can include a photosensitizer, which encompasses any agent that makes a cell more susceptible to a phototherapeutic, such as octylbromidezincphtametallo, Another class of chemo-adjuvants includes radiation sensitizers, which encompasses any agent that makes a cell more susceptible to a radiation or radio therapeutic. A radio-protective agent, which encompasses any agent that protects a cell from radiotherapy, can also be utilized as a chemo-adjuvant in a delivery system.

The chemo-adjuvant agent can be formulated as a controlled release, a triggered release, a sustained release, or a multistage release formulation. This can occur via the delivery vehicle being either a controlled release or sustained release vehicle, or can occur via the chemo-adjuvant being designed in a sustained release or controlled release formulation independently of the delivery vehicle formulation. A controlled release and/or sustained release formulation can allow the chemo-adjuvant to be effectively released over an extended period of time, such as over at least, 5 minutes, 30 minutes, 1 hour, 3, hours, 6 hours, 12 hours, 24 hours, 2 days, 4 days, or 1 week. The chemo-adjuvant agent can be spatially loaded on a delivery vehicle. For example, it can be located on the surface and/or at the interface of the core and shell structure and/or inside the delivery vehicle so as to establish the controlled release/sustained release profile. The chemo-adjuvant can be loaded in a macro or micro device for delivery into the body.

The chemo-adjuvant agent can be designed so as to be delivered according to a controlled release and/or sustained release profile independent of the design of the delivery vehicle. For example, the chemo-adjuvant can be encapsulated, conjugated, adsorbed, grafted, or complexed, or any combination of these with a secondary material so as to delay delivery of the chemo-adjuvant to a targeted cell.

The chemo-adjuvant can be provided in conjunction with a delivery vehicle to form the delivery system. As utilized herein, the term “delivery vehicle” generally refers to a molecule or a set of molecules in a form that can encapsulate, contain, or be conjugated to at least one chemo-adjuvant and that can be delivered to a location (e.g., a cell). The delivery vehicle can include, without limitation, cells, bacterium, viruses, lipids, polymers, proteins, amino acids, or their combinations. For example, the chemo-adjuvant can be covalently or non-covalently bonded to or encapsulated within a delivery vehicle. The chemo-adjuvant can generally be delivered more efficiently to the cell in when associated with the delivery vehicle than in the absence of the delivery vehicle.

The delivery vehicle can be of a size ranging from sub-nanometers to millimeters. Dimension of a particle or other delivery vehicle generally refers to the average diameter of a population of delivery vehicles with homogenous or non-homogenous sizes. In one embodiment the delivery vehicle can be particle that can be solid, porous, or hollow. In addition, a delivery vehicle can include one or more layers or coatings surrounding a core that can be hollow, porous, or solid. The coating can include, without limitation, cell walls, bacteria walls, lipids, polymers, proteins, amino acids, nucleic acids, or their combinations. For example, a delivery vehicle can be a nanoshell or a nanoring. When considering a layered delivery vehicle, each layer can have a unique composition and unique properties relative to the other layer(s), though this is not a requirement of a layered delivery vehicle.

A delivery vehicle can be a nanoparticle having a characteristic dimension of less than 1000 nanometers. In some embodiments, the average or mean diameter of the nanoparticles is less than about 300 nm, less than about 200 nm, less than about 150 nm, less than about 100 nm, less than about 50 nm, less than about 30 nm, less than about 10 nm, less than about 3 nm, less than about 1 nm, or any value or interval thereof. In other embodiments the delivery vehicle can be a microparticle having a characteristic dimension of equal to or greater than 1000 nanometers and less than 1000 micrometers (μm). For example, a microparticle can have a greatest dimension of less than 1000 μm. In some embodiments, a microparticle can have a greatest dimension of less than 100 μm. In certain embodiments, the average or mean diameter of a microparticle is greater than or about, 2 μm, or 4 μm.

The delivery vehicle is not limited to any particular shape and can have a shape ranging from a linear molecule, a branched molecule, a sphere, rod, needle shape, a cylinder, a cube, an amorphous shape, or any geometrical shape. In certain embodiments, the delivery vehicle comprises a homogenous or substantially homogenous mixture of molecules, and in other embodiments, the delivery vehicle can be non-homogenous.

Delivery vehicles can be made employing a variety of biodegradable polymers used for controlled release formulations, as are well known in the art. Derivatized biodegradable copolymers are also suitable for use in the present invention, including hydrophilic polymers (e.g., polyethylene glycol) attached to PLGA and the like or including side chain modifications. The delivery vehicle is not limited to biodegradable polymers, however, and it should be understood that the delivery vehicle can include a biodegradable or non-biodegradable synthetic or natural based component, metal, inorganic, lipid, protein, polymer, amino acid, nucleic acid, biological, nanocell, or any combination of these. In certain embodiments, the delivery system can include a homogenous mixture of delivery vehicles, and in other embodiments, the system comprises a nonhomogenous mixture of delivery vehicles.

Particles as may be utilized as delivery vehicles and methods for making suitable particles that can be employed include those described in, e.g., WO 97104747 to Dunn, et al., U.S. Pat. No. 6,007,845 to Domb et al., U.S. Pat. No. 5,578,325 to Domb et al., U.S. Pat. No. 5,543,158 to Gref, et al., U.S. Pat. No. 6,254,890 to Hirosue et al., U.S. Published Patent Application No. 2010/0144845 to Farokhzad, et al., U.S. Pat. No. 8,367,113 to Gu, et al., the complete disclosure of which are incorporated by reference herein. Composition and methods for making nanoparticles are also described in Farokhazad et al., “Targeted nanoparticle-aptamer bioconjugates for cancer chemotherapy in vivo,” PNAS 103(16):6315-6320 (Apr. 18, 2006), and Farokhazad et al., “Nanoparticle-Aptamer Bioconjugates: A New Approach for Targeting Prostate Cancer Cells,” Cancer Research 64, 7668-7672 (Nov. 1, 2004). Composition and methods for making nanoparticles are also described in Karnik et al. (Nano Lett. 2008 Sep; 8(9):2906-12. Epub 2008 Jul. 26.

In some embodiments, nanoparticles and microparticles can be built as aggregates of amphiphilic molecules that establish a hydrophobic core and expose hydrophilic moieties to the external media. For example, nanoparticles can be prepared using the water-in-oil-in-water solvent evaporation procedure (double emulsion method) as described previously, e.g., in Gref, R. et al., Science 263, 1600-1603 (1994). Nanoparticles can also be prepared as described in Farokhazad et al., “Targeted nanoparticle-aptamer bioconjugates for cancer chemotherapy in vivo,” PNAS 103(16):6315-6320 (Apr. 18, 2006).

A delivery vehicle may optionally comprise one or more dispersion media, surfactants, release-retarding ingredients, or other pharmaceutically acceptable excipients as are generally known in the art. For instance, a delivery vehicle may optionally comprise one or more plasticizers.

A delivery vehicle can include lipids. For example, liposomes can be utilized as a delivery vehicle, for instance cationic liposomes (e.g., DOTMA, DOPE, DC-cholesterol) or anionic liposomes. Liposomes can further comprise proteins to facilitate targeting a particular cell, if desired. Regarding liposomes for use as delivery vehicles, see, e.g., Brigham et al. Am. J. Resp. Cell. Mol. Biol. 1:95-100 (1989); Feigner et al. Proc. Natl. Acad. Sci USA 84:7413-7417 (1987); and U.S. Pat. No. 4,897,355 to Eppstein, et al., which is incorporated herein by reference.

The delivery vehicles can be produced using any known method for production. For example, methods including a bottom-up or top-down approach can be used. For example, particulate formulations can be formed by methods such as nanoprecipitation, self-assembly, directed assembly, layer-by-layer, printing, template based processes, flow focusing using fluidic channels, spray drying, single and double emulsion solvent evaporation, reverse micelle methods, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductors, conductive, magnetic, organic, and other nanoparticles have been described (Pellegrino at al., 2005, Small, 1:48; Murray at al., 2000, Ann. Rev. Mat. Sci., 30:545; and Trindade at al., 2001, Chem. Mat., 13:3843). In certain embodiments, particles are prepared by the nanoprecipitation process or spray drying. Conditions used in preparing particles may be altered to yield particles of a desired size or property (e.g., hydrophobicity, hydrophilicity, external morphology, “stickiness,” shape, etc.). The method of preparing the particle and the conditions (e.g., solvent, temperature, concentration, air flow rate, etc.) used may depend on the chemo-adjuvant and/or the therapeutic agent to be delivered. Methods for making microparticles for delivery of encapsulated agents are described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., b 36:766).

The delivery vehicle can optionally be conjugated to a variety of molecules and agents including targeting moieties and detecting moieties. By way of example, the delivery vehicle can be conjugated to molecules including an antibody, antibody fragment, peptide, protein, affibody, small molecules, macromolecules, RNA, DNA, or functional nucleic acids, such as an aptamer.

A delivery vehicle can be conjugated to a ligand that can provide a use to the delivery system, for instance as a targeting moiety for the delivery vehicle. A ligand is a substance or a composition or a molecule that is able to bind to and form a complex with a delivery vehicle or a chemo-adjuvant to serve a biological purpose. Ligands can include, without limitation, enzyme substrates, blockers, inhibitors, activators, and neurotransmitters. Radioligands are radioisotope labeled ligands, while fluorescent ligands are fluorescently tagged ligands; both can be considered as ligands and are often used as tracers for receptor biology and biochemistry studies.

In certain embodiments the delivery system can include functionalized micro- and/or nanoparticles as delivery vehicles. Functionalized particles can include, e.g., fluorescently labeled nanoparticles. Functionalized particles can also include targeting agents, which include targeting ligands. The targeting ligands can be a specific binding member for a cellular moiety. For instance, a targeting agent can specifically bind cell surface receptors such as CD133 on cancer stem cells, which can be expressed in U87 cells or other cancer stem cells.

As utilized herein, the term targeting agent generally refers to any agent that can specifically bind to a target. In certain embodiments, the targeting agent can be an antibody, a functional nucleic acid, peptide, peptide mimetic, affibody, diabody, glycoprotein, glycopeptide, proteoglycan, avimer, nanobody, adnectin, small molecule (e.g., Mw less than 2000 g/mol), carbohydrate, lipid, spiegelmer, and so forth. In certain embodiments, a single delivery vehicle can include more than one, such as 2, 3, 4, or 5 different targeting agents. A delivery system can optionally include a plurality of delivery vehicles that differ from one another according to targeting agents incorporated on/in the delivery vehicles. In certain embodiments, a targeting agent can specifically bind more than one antigen.

A targeting agent can bind a healthy cell, a cancer cell, extracellular matrix, a tissue, a protein, a carbohydrate, a glycoprotein, a proteoglycan, a lipid, a nucleic acid, an amino acid, or any other suitable target. In one embodiment, the targeting agent can bind a cell and trigger cellular internalization of the delivery vehicle carrying the chemo-adjuvant or the chemo-adjuvant following release from the delivery vehicle. A targeting agent can likewise trigger one or more of transcellular transport, endosome escape, surface binding, adsorbtion, conjugation, interactions, complexing, shielding, activation, etc. In certain embodiments, more than one targeting agent can be used, such as a second, or a third, or a fourth. These can be used in combination and can be different targeting agents, targeting either the same or a different target.

A targeting agent can specifically bind a target such as, and without limitation, CD133, CD3, CD4, CD8, CD19, CD20, CD22, CD34, CD40, CD79α, CD79α, CD79β CD79b, CD 140, CD 44, CD 24, CD 117, CD 15, Bmi-1, Notch, Sonic hedgehog/WntEGF, EGF, EGFR, FGF, FGFR, PDGF, PDGFR, PDGFRα, IGF, IGFR, nestin GFAP, β-tubulinlli, II-13, DIx2, PSA-NCAM, ABC transporters, BCRP1, SSEA1, MDR1, SOX1/2, c-MYC, OCT-4, BMI-1, Olig-2, MELK, PI3 kinase/Akt pathway, mTOR, MGMT, Mushashi1, ErbB receptor family member, HER2, HER3 HER4 receptor, a cell adhesion molecules, LFA-1, Mac 1, p150,95, VLA-4, ICAM-1, VCAM, αv/β3 integrin, CD11a, CD18, CD11b, a growth factor, VEGF, VEGFR, IgE, a blood group antigens, flk2/flt3 receptor, obesity (OB) receptor, mpI receptor, CTLA-4, protein C, BR3, c-met, tissue factor, β7, cell surface tumor associated antigen, transmembrane tumor-associated antigens (TAA), derivatives or variants.

A targeting agent or other useful ligand can be bound to a delivery vehicle according to standard chemistry. For example, a specific binding member for a cell surface receptor of a targeted cancer cell may be attached to the delivery vehicle using any of a variety of well-known techniques. For instance, covalent attachment of a targeting agent to the delivery vehicle (e.g., particles) may be accomplished using carboxylic, amino, aldehyde, bromoacetyl, iodoacetyl, thiol, epoxy and other reactive or linking functional groups, as well as residual free radicals and radical cations, through which a protein coupling reaction may be accomplished. A surface functional group may also be incorporated as a functionalized co-monomer because the surface of the delivery vehicle may contain a relatively high surface concentration of polar groups. In addition, although delivery vehicles can be functionalized after synthesis, the delivery vehicle may be capable of direct covalent linking with a targeting agent without the need for further modification. For example, in one embodiment, the first step of conjugation is activation of carboxylic groups on the surface of the delivery vehicle using carbodiimide. In the second step, the activated carboxylic acid groups are reacted with an amino group of an antibody or antibody fragment targeting agent to form an amide bond. The activation and/or antibody coupling may occur in a buffer, such as phosphate-buffered saline (PBS) (e.g., pH of 7.2) or 2-(N-morpholino)ethane sulfonic acid (MES) (e.g., pH of 5.3). The resulting delivery vehicle may then be contacted with ethanolamine, for instance, to block any remaining activated sites. Overall, this process forms a conjugated delivery vehicle, where the antibody is covalently attached to the delivery vehicle. Besides covalent bonding, other attachment techniques, such as physical adsorption, may also be utilized.

The delivery vehicle may be targeted to a particular cell type via antibodies, receptors, or receptor ligands. The following references are examples of the use of this technology to target specific proteins to tumor tissue: Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer lmmunol. Immunother., 35:421-425, (1992); Pietersz and McKenzie,_Immunolog. Reviews, 129:57-80, (1992); and Roffler, et al., Biochem. Pharmacol, 42:2062-2065, (1991)). These techniques can be used for a variety of specific cell types. Vehicles such as “stealth” and other antibody conjugated liposomes (including lipid mediated drug targeting to colonic carcinoma) can be utilized via receptor mediated targeting of DNA through cell specific ligands, lymphocyte directed tumor targeting, and highly specific therapeutic retroviral targeting of murine glioma cells in vivo. The following references are examples of methods for targeting specific proteins to tumor tissue (Hughes et al., Cancer Research, 49:6214-6220, (1989); and Litzinger and Huang, Biochimica et Biophysica Acta, 1104:179-187, (1992)). In one embodiment, receptors as may be targeted by a functionalized delivery vehicle can be involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration.

A delivery vehicle can be designed to provide the chemo-adjuvant according to a controlled release mechanism, in which the delivery vehicle can release the chemo-adjuvant over an extended period of time such as days at a controlled rate. For example, the delivery vehicle can release the chemo-adjuvant through diffusion, erosion, desorption, or some combination thereof. In one embodiment, a delivery vehicle can release a chemo-adjuvant according to a sustained release mechanism, in which the chemo-adjuvant is released for a short period of time such as hours. In such an embodiment, a treatment protocol may require repeated administration. A chemo-adjuvant can also be released from the delivery vehicle according to a triggered or activated release mechanism. A triggered or activated release mechanism refers to a system in which a chemo-adjuvant is released from the delivery vehicle through a stimulus such as change of pH, change of temperature, presence of enzymes, or other type of stimulus.

A delivery system can include one or more different types of delivery vehicles. For example, a delivery system can include a homogenous mixture of delivery vehicles or a non-homogenous mixture of delivery vehicles in which the vehicle mixture is composed of vehicles with different sizes, shapes components, etc.

A delivery system can include the delivery vehicle and the chemo-adjuvant associated with the delivery vehicle and one or more active agents, which can be therapeutic, for delivery to a subject. In certain embodiments, the active therapeutic agent and the delivery vehicle in conjunction with the chemo-adjuvant can be administered at the same time. Alternatively, the therapeutic agent and the delivery vehicle can be administered at different times, either the therapeutic agent first or the delivery vehicle first. For example, the therapeutic agent and the delivery system can be administered within 0.1 hours, 0.5 hours, 1 hours, 2 hours, 4 hours, 8 hours, 24 hours, 36 hours, 48 hours, or 72 hours, 84 hours or 96 hours of the other, or anytime following the administration of the therapeutic agent. Moreover, the therapeutic agent and the delivery vehicle can be administered in a similar fashion, e.g., both orally or intravenously, or can be administered according to two different delivery routes. In one embodiment, the therapeutic agent can be associated with the delivery vehicle. For example, a therapeutic agent can be encapsulated, covalently bonded to, non-covalently bonded to, contained within, or otherwise conjugated with the delivery vehicle that is also associated with a chemo-adjuvant,

The delivery system itself can include more than one delivery vehicle, such as 2, 3, 4, or 5 delivery vehicles, each delivery vehicle being associated with one or more chemo-adjuvant agents and/or one or more therapeutic agents.

The system can be formulated such that it can be administered to a subject, and in certain embodiments the system can be formulated for administration orally, pulmonary, intranasal, subcutaneously, intravenously, intraarterially, intrathecal, interventricular, intracavitary, directly into the brain parenchyma via diffusion or via convection enhanced delivery, locally, intratumor, topically, rectally, vaginally, intraperitoneally, or combinations thereof. In certain embodiments, the delivery system can be delivered through electroporation, through lipofection, or through calcium phosphate precipitation. The delivery mechanism chosen will depend in part on the type of cell targeted as well as the nature of the delivery vehicle and whether the delivery is occurring for example in vivo or in vitro, and for example, through ex vivo. For example, administration of a delivery system comprising a chemo-adjuvant in conjunction with a cationic liposome delivery vehicle can be administered to the blood afferent to a target organ or inhaled into the respiratory tract to target cells of the respiratory tract.

The delivery system can be formulated in a solid form, liquid form, syrup, suspension, or the like, generally depending upon the delivery route to be utilized. For example, the delivery system can be provided as a composition and can include an excipient, a wetting agent, an additive, or a propellant as are generally known in the art. The delivery system can be administered as a component of a microcapsule that can be targeted to specific cell types, such as macrophages, or where the diffusion of the compound or delivery of the compound from the microcapsule is designed for a specific rate or dosage.

The delivery system is designed for utilization in conjunction with a therapeutic agent. As utilized herein, the term therapeutic agent generally refers to any agent that reduces the amount of cancer cells in a subject or a testing protocol. A chemotherapeutic is any agent that that is a chemical and a therapeutic agent. For example, a chemotherapeutic agent can be an alkylating agent, antimetabolite, antibiotic, hormonal agent, hormonal inhibitor, anthracycline, plant alkaloid, topoisomerase inhibitor, tyrosine kinase inhibitor, phosphatase inhibitor, a retinoid, Golgi apparatus effector, oxydoreductase inhibitor, transferase inhibitor, hydrolase inhibitor, antifolate, antibiotic agent, taxane, antimitotic agent, differentiation agent, lyase inhibitor, isomerase inhibitor, ligase inhibitor, substrate inhibitor, endonuclease inhibitor, glycosylase inhibitor, PARP inhibitor, DNA alkylotransferase inhibitor, nitrosourea, methylhydrazines, steroid hormones, steroid hormone receptor, cytokine, growth factor, a retinoic acid derivative, a bone marrow growth factor, asparaginase, or derivatives of these, and/or where any of these therapeutics can be used alone or in combination with any other therapeutic disclosed herein.

Nitrosourea chemotherapeutics can include the chemicals carmustine, lomustine, semustine, or strepzotocin, or derivatives of these, among others. A methylhydrazine can include, for example, procarbazine or dacarbazine or derivatives of these. A steroid hormone can include a glucocorticoid, estrogen, progestin, androgen, or tetrahydrodesoxycaricosterone, or derivatives of these. An antimetabolite can include, for example, methotrexate, purine antagonists, mercaptopurine (6-MP), thioguanine (6-TG), fludarabine phosphate, cladribine (Leustatin®), pentostatin (Nipent®), pyrimidine antagonists, fluorouracil (5-FU), cytarabine (ARA-C), or azacitidine. A plant alkaloid can include vinblastine (Velban®), vincristine (Oncovin®), etoposide (VP-16, VePe-sid), teniposide (Vumon®), topotecan (Hycamtin®), irinotecan (Camptosar®), paclitaxel (Taxol®), or docetaxel (Taxotere®). An antibiotic can include, anthracyclines, doxorubicin (Adriamycin®, Rubex®, Doxil®), daunorubicin (DaunoXome®), dactinomycin (Cosmegen®), idarubincin (Idamycin®), plicamycin (Mithramycin®), mitomycin (Mutamycin®), or bleomycin (Blenoxane®). A hormonal agent can include tamoxifen (Nolvadex®) or flutamide (Eulexin®). A gonadotropin-releasing hormone agonist can include leuprolide or goserelin (Zoladex®). An aromatase Inhibitor can include aminoglutethimide or anastrozole (Arimidex®). Other examples of a therapeutic agent can include, without limitation, amsacrine, hydroxyurea (Hydrea®), asparaginase (Elspar®), mitoxantrone (Novantrone®), mitotane, or amifostine (Ethyol®).

The therapeutic agent can be an activated prodrug, such as, and without limitation, light activated prodrugs such as photofrin, levulan, metvixia, etc.; enzyme activated prodrugs such as 5-fluorocytosine; methotrexate alanine, doxorubicin phosphate etc.

In one embodiment the therapeutic agent can be an alkylating agent, which can include subclasses such as a classical alkylating agent, nonclassical alkylating agent, alkylating mimetics, proalkylating agent, dialkylating agent, or acylfulvene alkylating agent, polyfunctional alkylating agents. A polyfunctional alkylating agent can include bis(chloroethyl)amine, ethylenimine, and nitrosoureas. Alkylating agents include compounds such as but not limited to: carmustine (BCNU), lomustine (CCNU), semustine (methyl CCNU), cyclophosphamide (Cytoxan®), fosfamide, mechlorethamine, melphalan (Alkeran®), chlorambucil (Leukeran®), thiopeta (Thioplex®), busulfan (Myleran®), procarbazine (Matulane®), dacarbazine (DTIC), altretamine (Hexalen®), and cisplatin (Platinol®).

Classical alkylating agents can include cisplatin, carboplatin, oxaliplatin, nitrogen mustards, mechlorethamine, cyclophosphamide, chlorambucil, ifosfamide, methyl bis(B-chloroethyl)emine hydrochloride (mechlorethamine, mustine), tris(B-chloroethy)amine hydrochloride, uramustine or uracil mustard, melphalan, ifosfamide, nitrosoureas, carmustine, lomustine, streptozocin, alkyl sulfonates, busulfan, and thiotepa. Nonclassical alkylating agents can include procarbazine, altretamine, tetrazines, dacarbazine, mitozolomide, fotemustine, and temozolomide.

An alkylating-mimetic can include platinum mimetics, platinum, cisplatin, carboplatin, nedaplatin, oxaliplatin, satraplatin, and triplatin tetranitrate. An example of a proalkyating agent is cyclophospahmaide. A dialkyating agent can include busulfan (methanesulfonate diester of 1,4-butanediol).

The therapeutic agent can be provided in any suitable form, generally depending upon the desired administration route. For example, the therapeutic agent can be proved as a salt, a solid, a liquid, a suspension, an aggregate, or a gel. The therapeutic agent can be active against one or multiple pathways such as dasatinib, imatinib, lapatinib, nilotinib, sorefinib, sunitinib, and derivatives of these, and/or where any of these limitations can be used alone or in combination with any other limitation disclosed herein.

In accordance with one embodiment, the delivery system and/or delivery vehicle can be provided in conjunction with a local drug delivery apparatus. A local drug delivery apparatus can be a medical device for implantation into a treatment site of a living organism and can include at least one delivery vehicle and/or a therapeutic agent in a therapeutic dosage releasably affixed to the medical device. A local delivery apparatus can include a material for preventing the delivery vehicle and/or the therapeutic agent from separating from the medical device prior to implantation of the medical device at the treatment site, the material being affixed to the medical device or a component of the delivery system.

A delivery vehicle and/or a therapeutic agent may be affixed to any number of medical devices. For example, the delivery vehicles and systems can be associated with or fixed with pumps, catheters, or implants. A delivery system may be affixed to minimize or substantially eliminate the biological organism's reaction to the introduction of the medical device utilized to treat a separate condition. For example, stents, catheters, implants, balloons, self-expandable or nor, degradable or not, can be utilized in conjunction with the delivery system.

The components of the delivery system can be administered in vivo by use of a pharmaceutically acceptable carrier in the form of a composition. By “pharmaceutically acceptable” is meant a material that is not biologically or otherwise undesirable, i.e., the material may be administered to a subject, along with the components of the delivery system, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of pharmaceutically-acceptable carriers include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the delivery system, which matrices are in the form of shaped articles, e.g., films. It will be apparent to those persons skilled in the art that certain carriers may be more preferable depending upon, for instance, the route of administration and concentration of composition being administered.

Pharmaceutical compositions for use in conjunction with the delivery system may include carriers, thickeners, diluents, buffers, preservatives, surface active agents and the like in addition to the delivery vehicle and/or the therapeutic agent. Pharmaceutical compositions may also include one or more active ingredients such as antimicrobial agents, anti-inflammatory agents, anesthetics, and the like.

A pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The delivery system can be administered intravenously, intraperitoneally, intramuscularly, subcutaneously, intracavity, or transdermally.

Parenteral administration, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained.

Preparations for parenteral administration can include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral carriers include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

A composition may be administered by use of a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The delivery system can be used to treat any disease where uncontrolled cellular proliferation occurs such as cancers. In so treating, the delivery vehicles can be delivered to the subject, for example, wherein the subject is in need of a treatment for cancer. The administration can occur as described herein, or as otherwise understood in the art. The delivery vehicles can be delivered with a therapeutic agent, before a therapeutic agent, or after a therapeutic agent. When delivered at the same time, it is understood that the therapeutic agent can either be within the delivery vehicle containing the chemo-adjuvant, or it can be in a separate delivery vehicle.

The delivery system can be used to treat any disease where uncontrolled cellular proliferation occurs such as cancers. A non-limiting list of cancers as may be treated by use of the delivery system includes glioblastoma, medulloblastoma, leukemia, Hodgkins lymphoma, non-Hodgkins lymphoma, carcinoma, sarcoma, myeloma, prostate cancer, bladder cancer, breast cancer, kidney cancer, pancreatic cancer, anal cancer, oseophegal cancer, colon cancer, skin cancer, bilary cancer, stomach cancer, head and neck cancer, solid carcinoma, squamous cell carcinoma, adenocarcinoma, glioma, high grade glioma, blastoma, neuroblastoma, plasmacytoma, histiocytoma, melanoma, adenoma, hypoxic tumour, myeloma, AIDS-related lymphoma or AIDS-related sarcoma, or metastatic cancer, B cell lymphoma, T cell lymphoma, mycosis fungoides, Hodgkin's Disease, myeloid leukemia, brain cancer, nervous system cancer, squamous cell carcinoma of head and neck, lung cancer, small cell lung cancer, non-small cell lung cancer, ovarian cancer, pancreatic cancer, prostate cancer, skin cancer, liver cancer, melanoma, squamous cell carcinoma of the mouth, squamous cell carcinoma of the throat, squamous cell carcinoma of the larynx, squamous cell carcinoma of the lung, colon cancer, cervical cancer, cervical carcinoma, epithelial cancer, renal cancer, genitourinary cancer, pulmonary cancer, esophageal carcinoma, large bowel cancer, hematopoietic cancer, testicular cancer, rectal cancer, prostatic cancer, or pancreatic cancer.

The delivery system may also be used for the treatment of precancer conditions such as cervical and anal dysplasias, other dysplasias, severe dysplasias, hyperplasias, atypical hyperplasias, and neoplasias. The delivery system may also be administered prophylactically to subjects who are at risk for a cancer as described herein.

A subject as may be treated by use of the delivery system can include any individual in need of such treatment. Thus, the subject can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human. The subject can also be a non-human.

Effective dosages and schedules for administering the delivery system may be determined empirically, and making such determinations is within the skill in the art. The dosage ranges for the administration of the delivery system are those large enough to produce the desired effect. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products.

Following administration of the delivery system for treating, inhibiting, or preventing a cancer, the efficacy of the therapeutic agent can be assessed in various ways well known to the skilled practitioner. For instance, one of ordinary skill in the art will understand that a therapeutic agent delivered in conjunction with the chemo-adjuvant is efficacious in treating or inhibiting a cancer in a subject by observing that the therapeutic agent reduces the cancer cell load or prevents a further increase in cancer cell load. Cancer cell loads can be measured by methods that are known in the art, for example, using polymerase chain reaction assays to detect the presence of certain cancer cell nucleic acids or identification of certain cancer cell markers in the blood using, for example, an antibody assay to detect the presence of the markers in a sample (e.g., but not limited to, blood) from a subject or patient, or by measuring the level of circulating cancer cell antibody levels in the patient.

The present disclosure may be better understood with reference to the examples, set forth below. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### EXAMPLE

It was demonstrated that cells labeled with several different types of progenitor markers found in and around a glioblastoma recurrence are actively dividing. Confocal photomicrographs of left frontal human glioblastoma recurrence are shown in FIG. 2. As shown in FIG. 2A, CD 133+ cells were more abundant closer to the subventricular zone (SVZ) nearest to the dorsolateral horn of the left lateral ventricle, (FIG. 2A). The border between the glioblastoma recurrence and the subventricular zone is demarcated by a line in FIG. 2A and magnified on the images on the right (FIG. 2B and FIG. 2C). Co-labeling GFAP+/CD 133+ cells were seen in this region.

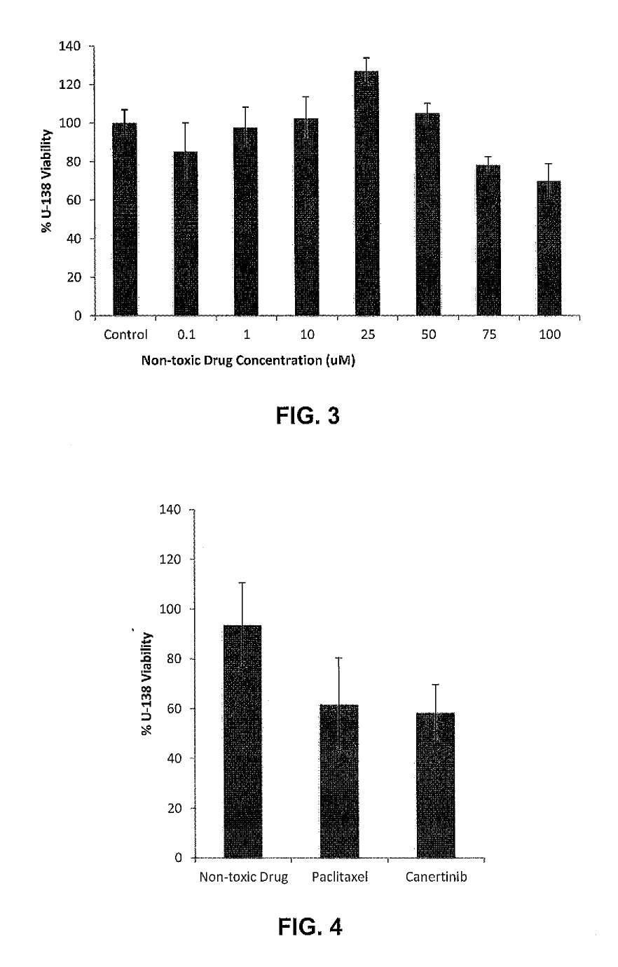

Various drug combinations were incubated with U138 cells in cell culture media and the cells were then washed with PBS and added fresh media. MTT (MTT assay=(3-(4,5-Dimethylthiazol-2-yl)-2,5-diphenyltetrazolium bromide, a yellow tetrazole) assays were carried out to quantify cell viability. Cytotoxicity assays were carried out and the results are shown in FIGS. 3-6. FIG. 3 illustrates cell viability following incubation with various concentrations of the non-toxic chemo adjuvant O6-BG. As can be seen, there is some effect on cell viability at very high concentrations of the non-toxic chemo-adjuvant. FIG. 4 illustrates the results of a comparison of cell viability for U138 cells following incubation with the non-toxic chemo-adjuvant (1000 higher concentration than toxic therapeutic agent; 100 uM) and with two different toxic therapeutic agents, including paclitaxel (100 nM) and canertinib (100 nM). As expected, cell viability is lower with the therapeutic agents.

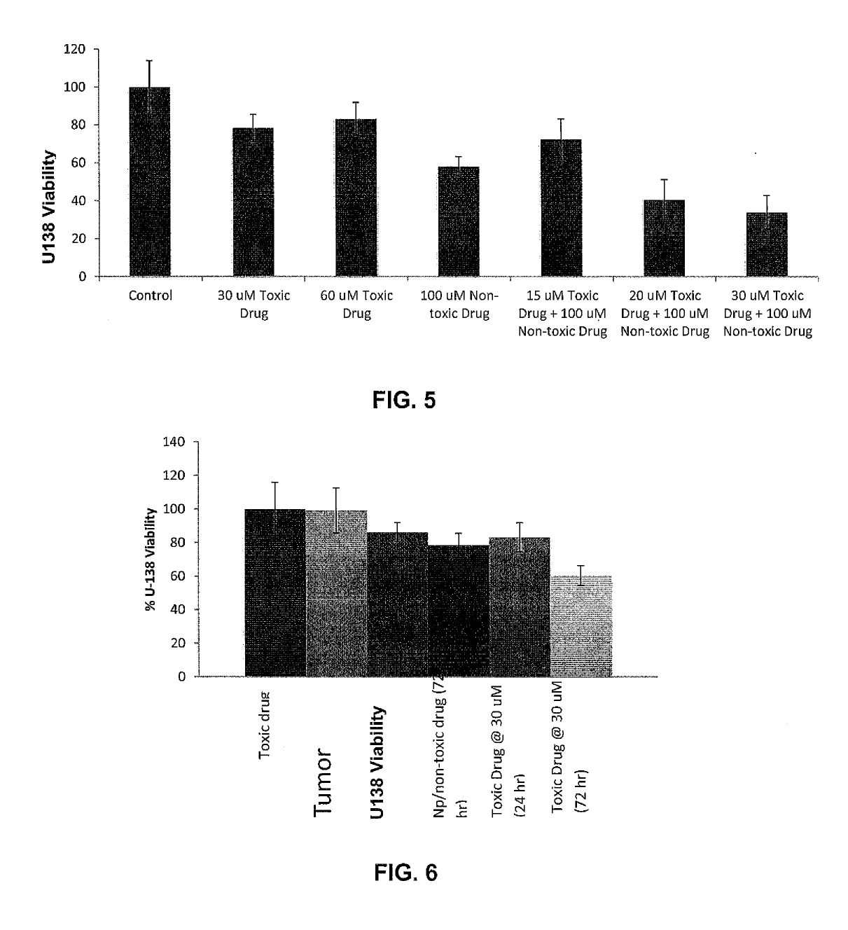

FIG. 5 illustrates the viability results for various combinations of a non-toxic chemo-adjuvant (O6-BG) and a toxic therapeutic agent (Temozolomide. In FIG. 6, results are shown following incubation of various concentrations of the toxic therapeutic agent temozolomide with and without nanoparticles (rip) loaded with the non-toxic chemo-adjuvant. The results illustrate that free O6-BG (100 μg/ml) was not cytotoxic at the tested concentrations. O6-BG loaded (100 μg/ml) nanoparticles combined with temozolomide (30 μM) was more cytotoxic than temozolomide (60 uM) showing that O6-BG loaded nanoparticles can efficiently increase TMZ-mediated cytotoxicity compared to TMZ, while reducing TMZ dosage.

Two functionalized nanoparticles were made with targeting ligands at various surface densities to assess their ability to efficiently bind and internalize to U87 and its subpopulation of CD133+ tumor cells. To determine the uptake of nanoparticle formulations flow cytometry, microplate analysis, and confocal fluorescent microscopy were used. Specific uptake was analyzed using blocking experiments with free targeting ligand incubated together with various nanoparticle formulations, including non-targeted nanoparticles as a negative control. Each experiment included 5 repeats for proper statistical analysis.

Following the ACURO guidelines and the disclosed approved protocol for brain tumor induction (AUP 2009-045) human intracranial glioblastoma xenografts using a U138 cell line were introduced into balb/c nude mice. To establish intracranial gliomas in mice, U138 cells were suspended in a solution of double strength RPMI 1640 containing 1% agarose and kept at room temperature until injected in adult mice. Mice were anesthetized with ketamine/xylazine (i.p., ˜4.5 mg/0.3 mg per 25 gm mouse) and immobilized in a stereotactic frame. A linear skin incision was made over the bregma, and a 1-mm diameter burrhole was drilled into the skull 3 mm posterior and 3 mm lateral to the bregma. The tumor cell suspension (10 ul/2×10̂5 cells) was injected using a Hamilton syringe with a 27-gauge disposable needle through the burr hole. The needle was covered by a plastic sleeve permitting an injection depth of 3.5 mm from the outer table of bone. The needle was withdrawn 30 seconds after injection of the cell suspension, and the bone hole was covered with sterile bone wax to prevent leakage of cerebrospinal fluid. The skin was closed with a skin clip. Tumor growth was allowed to reach a size of ˜1-mm (˜10 days post-injection). Intravenously fluorescently labeled non targeted and targeted nanoparticles were injected to determine the targeting efficiency and tissue distribution of nanoparticle formulations into the brain and glioblastoma tissue using a non-invasive imaging system. The fluorescent signal from the brain, heart, and tibia bone was normaiized based on the mass of tissue and signal in the liver. Next, the tissues were fixed in a solution of 4% paraformaldehyde, section (5 um), and imaged using confocal microscopy. This analysis was repeated to increase the statistical analysis.

Balb/c nude mice were used for in vivo biodistribution and tissue distribution studies. 5 animals for each nanoparticle formulation were used. At the end point of biodistribution experiments (˜2 days), the brain, liver, tibia bone, and heart were further analyzed by fluorescence microscopy. An additional group of 5 mice were used as a negative control. In certain studies, a total number of mice required was estimated to be ˜30 based on the factor of 1.2 to account for possible sickness and morbidity during the experimental period.

The number of repeats used for the biodistribution experiments was 5 animals to provide sufficient statistical analysis. Signal from each tissue was quantified using a non-invasive small animal imaging system and normalized with the mass of each organ. The signal of the liver was used as a positive control organ for semi-quantitative analysis across each animal and formulation. Data was plotted using standard deviation and an Anova-test was used to identify statistical differences with p<0.1 and p<0.05.

What have been described above are examples. It is, of course, not possible to describe every conceivable combination of components or methodologies for purposes of describing the subject matter, but one of ordinary skill in the art will recognize that many further combinations and permutations of the subject matter are possible. Accordingly, the disclosure is intended to embrace all such alterations, modifications, and variations that fall within the scope of this application, including any appended claims.

What is claimed is:

1. A delivery system, comprising:
a chemo-adjuvant, the chemo-adjuvant enhancing the efficacy of a therapeutic agent in treatment of a cancer; and
a delivery vehicle, the chemo-adjuvant being provided in conjunction with the delivery vehicle.

2. The delivery system of claim 1, wherein the delivery vehicle comprises a microparticle or a nanoparticle.

3. The delivery system of claim 1, wherein the chemo-adjuvant is covalently bonded to the delivery vehicle, is non-covalently bonded to the delivery vehicle, or is encapsulated within the delivery vehicle.

4. The delivery system of claim 1, wherein the delivery vehicle is biodegradable.

5. The delivery system of claim 1, further comprising a targeting agent provided in conjunction with the delivery vehicle.

6. The delivery system of claim 5, wherein the targeting agent comprises a specific binding member for a cellular moiety.

7. The delivery system of claim 5, wherein the targeting agent specifically binds CD133, CD3, CD4, CD8, CD19, CD20, CD22, CD34, CD40, CD79α (CD79α and CD79β (CD79b), CD 140, CD 44, CD 24, CD 117, CD 15, Bmi-1, Notch, Sonic hedgehog/WntEGF, EGF, EGFR, FGF, FGFR, PDGF, PDGFR, PDGFRα, IGF, IGFR, nestin GFAP, β-tubulin III, II-13, DIx2, PSA-NCAM, ABC transporters, BCRP1, SSEA1, MDR1, SOX1/2, c-MYC, OCT-4, BMI-1, Olig-2, and MELK, PI3 kinase/Akt pathway, mTOR, MGMT, Mushashi1, ErbB receptor family member, HER2, HER3 HER4 receptor, a cell adhesion molecule, LFA-1, Mac 1, p150,95, VLA-4, ICAM-1, VCAM, αv/β3 integrin, CD11a, CD18, CD11b, a growth factor, VEGF, VEGFR, IgE, blood group antigens, flk2/flt3 receptor, obesity (OB) receptor, mpI receptor, CTLA-4, protein C, BR3, c-met, tissue factor, β7, cell surface tumor associated antigen, transmembrane tumor-associated antigens (TAA) or any surface or intracellular receptor, ligand, binding site, DNA, RNA or protein targetable.

8. The delivery system of claim 1, wherein the chemo-adjuvant is a chemo-sensitizer, radiation sensitizer, photosensitizer, detoxification agent, chemo-enhancing agent, chemo-protective agent, radio-protective agent, an MGMT inhibitor, a DNA repair inhibitor, leucovorin, any derivatives of these, or any combinations of these.

9. The delivery system of claim 1, wherein the chemo-adjuvant is non-toxic.

10. The delivery system of claim 1, wherein the chemo-adjuvant is an O6 substituted compound.

11. The delivery system of claim 1, further comprising a second chemo-adjuvant.

12. The delivery system of claim 1, further comprising a therapeutic agent.

13. The delivery system of claim 12, wherein the therapeutic agent is an alkylating agent, antimetabolite, antibiotic, hormonal agent, hormonal inhibitor, anthracycline, plant alkaloid, topoisomerase inhibitor, monoclonal antibody, tyrosine kinase inhibitor, phosphatase inhibitor, steroid hormone receptor, a retinoid, Golgi apparatus effector, oxydoreductase inhibitor, transferase inhibitor, hydrolase inhibitor, antifolate, antibiotic agent, taxane, antimitotic agent, differentiation agent, lyase inhibitor, isomerase inhibitor, ligase inhibitor, substrate inhibitor, endonuclease inhibitor, glycosylase inhibitor, PARP inhibitor, DNA alkylotransferase inhibitor, nitrosourea, methylhydrazine, steroid hormones, cytokine, growth factor, a retinoic acid derivative, a bone marrow growth factor, Gonadotropin-Releasing Hormone Agonist, Aromatase Inhibitor, or derivatives of these.

14. The delivery system of claim 12, wherein the therapeutic agent is an alkylating agent.

15. The delivery system of claim 14, wherein the alkylating agent is temozolomide.

16. The delivery system of claim 14, wherein the alkylating agent is a classical alkylating agent, a nonclassical alkylating agent, an alkylating mimetic, a proalkylating agent, a dialkylating agent, an acylfulvene alkylating agent, a polyfunctional alkylating agents or derivatives of these.

17. The delivery system of claim 14, wherein the therapeutic agent is provided in conjunction with the delivery vehicle.

18. The delivery system of claim 1, wherein the system is formulated in a solid form, in a liquid form, or as a suspension.

19. The delivery system of claim 1, wherein the system is in the form of a composition and comprises an excipient, a wetting agent, an additive, or a propellant.

20. A method of reducing the amount of cancer cells in an area, the method comprising administering a delivery system to the area, the area comprising cancer cells, the delivery system comprising a chemo-adjuvant, the chemo-adjuvant enhancing the efficacy of a therapeutic agent in reducing the amount of the cancer cells in the area, the delivery system further comprising a delivery vehicle, the chemo-adjuvant being provided in conjunction with the delivery vehicle.

21. The method of claim 20, wherein the chemo-adjuvant is released from the delivery vehicle in the area over an extended period of time.

22. The method of claim 20, wherein the chemo-adjuvant is released from the delivery vehicle according to a controlled release mechanism, a triggered or activated release mechanism, or a sustained release mechanism.

23. The method of claim 20, the delivery system further comprising a targeting agent in conjunction with the delivery vehicle, the targeting agent specifically binding a structure in the area.

24. The method of claim 23, wherein the structure is a cancer cell.

25. The method of claim 20, further comprising administering a therapeutic agent to the area.

26. The method of claim 25, wherein the therapeutic agent and the delivery system are administered to the area at the same time.

27. The method of claim 25, wherein the therapeutic agent and the delivery system are administered at different times.

28. The method of claim 20, wherein the method is a cancer treatment method and the delivery system is administered to a subject.

29. The method of claim 28, wherein the delivery system is administered to the subject orally, pulmonary, intranasal, subcutaneously, intravenously, intraarterially, intrathecal, interventricular, intracavitary, directly into the brain parenchyma via diffusion or via convection enhanced delivery, locally, intratumor, topically, rectally, vaginally, intraperitoneally.

30. The method of claim 28, further comprising administering a therapeutic agent to the subject.

31. The method of claim 30, wherein the therapeutic agent and the delivery system are administered to the subject through different routes.

32. The method of claim 30, wherein the cancer is a glioblastoma.
